(19) United States
(12) Patent Application Publication
(10) Pub. No.: US 2019/0254942 A1
(43) Pub. Date: Aug. 22, 2019

(54) Microbiologically Stable Surfactant-Containing Formulation

(71) Applicant: Evonik Degussa GmbH, Essen (DE)

(72) Inventors: Jan Jänichen, Hamburg (DE); Wilfried Petersen, Hamburg (DE)

(73) Assignee: Evonik Degussa GmbH, Essen (DE)

(21) Appl. No.: 16/301,484

(22) PCT Filed: May 18, 2017
(86) PCT No.: PCT/EP2017/061940

§ 371 Date: Nov. 14, 2018

(30) Foreign Application Priority Data
May 23, 2016 (EP) 16170893.8

(51) Int. Cl. A61K 8/34; A61K 8/02; A61Q 5/02; A61Q 19/10

(52) U.S. Cl. CPC A61K 8/345; A61K 8/02; A61K 2800/74; A61Q 19/10; A61K 2800/30; A61Q 5/02

(57) ABSTRACT

The invention provides an aqueous formulation comprising:
a) at least one anionic surfactant selected from the group of alkyl sulphates, alkyl ether sulphates and/or acyl glutamates,
b) at least one amphoteric surfactant selected from the group of alkyl betaines and/or amidoalkyl betaines,
c) 2-methyl-1,3-propanediol.

It is provided according to the invention that
d) said formulation has a pH of 6 to 10,
e) comprises no further preservatives for microbiological stabilization selected from the group consisting of:
e1) authorized preservatives cited in Annex V of Regulation (EC) No. 1223/2009, and
e2) multifunctional additives selected from the group of linear or branched, saturated or unsaturated aliphatic or aromatic hydroxamic acids having a number of 6-16 carbon atoms and aromatic alcohols of the general structure

Ar—(CH2)n-OH

where n=2-4.

### FIELD OF THE INVENTION

The invention relates to an aqueous formulation according to the generic terms of claim 1.

### PRIOR ART

Preservatives are used in surfactant-containing products, particularly in cosmetic products, in which the presence of water and biologically utilizable materials such as fats, oils and surface-active compounds form an ideal matrix for the growth of bacteria, yeasts and fungi.

The control of the growth of microorganisms in these products is required in order to maintain both efficacy and appearance of the formulations and also to ensure the safety of consumers.

It is known to those skilled in the art that microorganisms flourish in the aqueous phase and avoid the lipophilic environment. To control microorganisms in cosmetic products, therefore, not only is the antimicrobial efficacy of a preservative system used for this purpose of importance, but also its concentration in the aqueous phase. Many lipophilic structures having in principle good antimicrobial properties due to their lipophilic structure are therefore only of limited suitability for preserving cosmetic products.

Prior to the end of the millenium, the preservation particularly of cosmetic and dermatological products was given very little attention. The costs and efficiency of a preservative were crucial for the manufacturer of the products and often the lack of production hygiene was compensated for by the excessive use of highly active preservatives. In addition, the criteria for determining the microbiological stability were so demanding that excessive amounts of highly active and chemically acting preservatives such as formaldehyde/formaldehyde releasers and/or isothiazolinones found their way into cosmetic and dermatological mixtures. As a consequence of their excessive use, a growing number of irritations and sensitizations were observed in consumers of cosmetic products which could be demonstrably traced back to these chemically acting preservatives. As a consequence of this finding, many of the traditional preservatives, particularly for use in cosmetic formulations, were therefore limited by legislation, or their public reputation suffered significantly.

For the developer of surfactant-containing cosmetic products in particular, the increasing limitations in the use of agents for microbiological stabilization of such products represent a major challenge. Its legal obligation for marketing exclusively microbiologically safe products require the consumer to resort only to selected traditional preservatives on the one hand or on the other hand at best to dispense with such systems. Furthermore, such a developer is confronted conceptually with many framework conditions such as the marketing concept or the pH of the formulation which conflict with the use of certain preservation systems.

Surfactant-containing products are not subject to any pH restrictions such that they can usually be formulated in the acidic pH range between pH 4.5-5.5 and can be successfully stabilized against microbial contamination using organic acids such as benzoic acid or sorbic acid. If the product concept, however, requires pH values above this level or those which are instead in a neutral medium, organic acids lose their effectiveness. Making the situation worse in such pH conditions is that numerous pH-independent preservation systems, such as, for example, phenoxyethanol, according to experience have only a very limited efficacy in surfactant-containing formulations. Whereas in emulsion-based product types the distribution of the preservation systems depends on their individual solubility in the oil or water phase, the situation in surfactant-based formulations is more complex. Such systems should have good cleansing power and their viscosity can be conveniently modified in order to produce ready-to-use products. These requirements are accomplished preferably with the aid of anionic and amphoteric surfactant components which are capable of forming charged micellar structures on the surface. Their packing density has an effect on the motility of the micelles and therefore determines the rheological properties of the formulation. However, the pH-independent preservation systems mentioned such as, for example, phenoxyethanol, can be enclosed in such micelles. They are therefore removed from the aqueous phase and cannot provide sufficient protection against microorganisms potentially present.

This applies particularly, as known to those skilled in the art, to surfactant systems based on combinations of anionic surfactants such as lauryl sulphates, lauryl ether sulphates or acyl glutamates with acylamidoalkyl betaines or alkyl betaines.

Consequently, alternatives for secure stabilization of, for example, almost pH-neutral surfactant formulations with preservatives acceptable to the public, are lacking to the developer.

The object of the present invention is to close the above-described loophole of efficient preservation systems for surfactant-containing products in general and for cosmetic products for cleansing skin and hair in particular and to make such products available to the developer having reliable antimicrobially effective systems for such product concepts.

### DESCRIPTION OF THE INVENTION

The invention achieves this object by the subject matter of the independent claim. Advantageous configurations are described in the dependent claims.

The invention therefore provides an aqueous formulation comprising:

a) at least one anionic surfactant selected from the group of alkyl sulphates, alkyl ether sulphates and/or acyl glutamates,

b) at least one amphoteric surfactant selected from the group of alkyl betaines and/or amidoalkyl betaines,

c) 2-methyl-1,3-propanediol,

characterized in that

d) said formulation has a pH of 6 to 10,

e) comprises no further preservatives for microbiological stabilization selected from the group consisting of:

e1) authorized preservatives cited in Annex V of Regulation (EC) No. 1223/2009, and

e2) multifunctional additives selected from the group of linear or branched, saturated or unsaturated aliphatic or aromatic hydroxamic acids having a number of 6-16 carbon atoms and aromatic alcohols of the general structure

Ar—(CH2)n-OH

where n=2-4.

The invention has recognized that 2-methyl-1,3-propanediol, surprisingly, has a sufficient biocidal effect as the sole preservative in a claimed composition in the mildly alkaline range of pH 6 to 10 without the need for further preservatives.

The aqueous formulation according to the invention is monophasic, consisting therefore exclusively of a water phase. It does not take the form of an emulsion.

In accordance with the invention, therefore, the presence of further preservatives is in particular excluded according to feature e); preferably no further preservatives are present.

Although 2-methyl-1,3-propanediol is already known as a constituent of cosmetic formulations in the prior art, it was unknown and surprising that this substance displays sufficient effect as sole preservative in the claimed composition.

2-Methyl-1,3-propanediol has been marketed for many years by Lyondell under the tradename MP-Diol® Glycol in various industry sectors. For use in the cosmetic industry, it has been advertised by Lyondell predominantly due to its solubilizing properties both for hydrophilic and lipophilic substances and for skin moisturizing. Moreover, 2-methyl-1,3-propanediol (INCI: Methylpropanediol) is also said to have odour-reinforcing properties in scent-containing formulations (source: Lyondellbassell, MP-Diol® Glycol, “A Product for the Personal Care Industry”, 2011).

The antimicrobial properties of 2-methyl-1,3-propanediol have also been investigated by Lyondell. The minimum inhibitory concentrations (MIC) against the microbes tested were between 0.5 and 50%. For relevant cosmetic applications and in a classic cosmetic contamination test according to Pharm. Eur. 2014, 5.1.3, microorganisms tested detected MIC values between 10 and 20% and minimum kill concentrations between 20 and 40% (source: Lyondell Chemical, “Antimicrobial Screen”, 2000). Based on these results, MP-Diol® Glycol was said to support or enhance properties of preservatives, but sole use for stabilizing cosmetic products was not suggested (source: Lyondell Chemical, “Personal Care Formulation Guide Screen”, 1998).

The enhancing effect of 2-methyl-1,3-propanediol on the preservative phenoxyethanol is disclosed in JP-A 11-279023.

The use of 2-methyl-1,3-propanediol in products for cleansing skin is described in various patent documents. EP 1334715 B2 discloses the combination of 2-methyl-1,3-propanediol with oils for preparing cleansing emulsions in the presence of further preservatives.

FR 2780283 A1 describes aqueous formulations which do not include any of the preservation systems mentioned in Annex V of the cosmetic guidelines and their microbiological stabilization is based on a combination of 2-methyl-1,3-propanediol and a complexing agent. However, the working examples mentioned in FR 2780283 A1 represent emulsions, do not feature any of the surfactants mentioned in the present application and were formulated in weakly acidic media (pH 5). Moreover, the results of microbiological investigations given again in Tables 3 and 4 show that 2-methyl-1,3-propanediol alone does not have sufficient antimicrobial effect and the presence of the complexing agent Na4EDTA is imperatively necessary.

For many years Dr. Straetmans GmbH has provided a multifunctional preservation system under the tradename Dermosoft® OMP, which comprises 2-methyl-1,3-propanediol and which is supplied for general use in cosmetic formulations including those for cleansing skin and hair. The antimicrobial principle of this blend, described in EP 0524548 B1, is based in the synergistic effect of a combination of 1,2-diols and phenylalkanols, which is enhanced by 2-methyl-1,3-propanediol.

Under the names Spectrastat® G and Spectrastat® H, the company Inolex provides multifunctional preservation systems comprising 2-methyl-1,3-propanediol in addition to further constituents and which are supplied also for surfactant-containing cosmetic formulations in the neutral pH range. The distributor of this blend on its internet site (http://inolex.com/PC/Products/Preservation-Systems/Spectrastat-Series/Spectrastat-G and -H) ascribes the antimicrobial effect of the blend supplied to the raw materials caprylohydroxamic acid and glyceryl caprylate or ethylhexylglycerin also present in the mixtures. Indications of an effect without the two additional components are not presented.

In summary, 2-methyl-1,3-propanediol according to the current prior art can be considered accordingly as a prevalent raw material for use in cosmetic products, whose supporting function in the antimicrobial effect of preservation systems in various product types has been described. The possibility of using 2-methyl-1,3-propanediol as sole constituent for the microbiological stabilization of an almost pH neutral or mildly alkaline and surfactant-containing formulation is surprising and has not been described to date.

In an alternative embodiment, the formulation according to the invention comprises as component a) at least one anionic surfactant selected from the group of alkyl sulphates and/or acyl glutamates,

and as component b) at least one amphoteric surfactant selected from the group of alkyl betaines and/or amidoalkyl betaines.

In a further alternative embodiment, the formulation according to the invention comprises as

component a) at least one anionic surfactant selected from the group of alkyl sulphates, alkyl ether sulphates and/or acyl glutamates,

and as component b) at least one amphoteric surfactant selected from the group of alkyl betaines.

Surfactant-containing formulations according to the invention comprise a combination of anionic and amphoteric surfactants as active washing components. The anionic components are selected from the group of alkyl sulphates, alkyl ether sulphates or acyl glutamates. The proportion of these surfactants is typically about 0.1 to 30, preferably 2 to 25 and especially 5 to 20% by weight, wherein the percentages by weight refer to the total formulation.

The alkyl sulphates described in the context of the present invention may be obtained by sulfation of one or more fatty alcohols of chain length 8-16 carbon atoms and subsequent neutralization. Accordingly, they have the general structure:

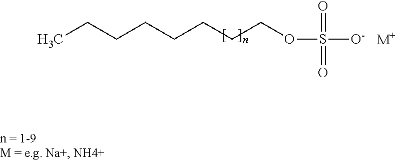

As representatives of this group, examples include, but are not limited to, the following raw materials with their INCI names: Sodium Lauryl Sulfate, Ammonium Lauryl Sulfate, Sodium Coco Sulfate.

If fatty alcohols having a chain length of 8-16 carbon atoms are firstly reacted with 1-4 moles of ethylene oxide and subsequently sulphated, the claimed alkyl ether sulphates after neutralization are produced of the general structure:

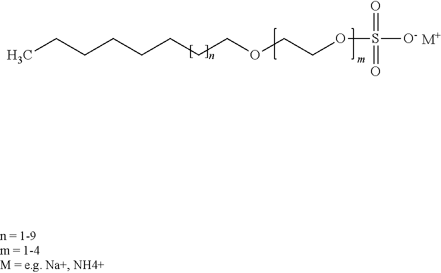

As representatives of this group, examples include, but are not limited to, the following raw materials with their INCI names: Sodium Laureth Sulfate, Ammonium Laureth Sulfate, Sodium Coceth Sulfate.

Acyl glutamates according to the invention may be prepared from fatty acids with a chain length of 8-16 carbon atoms and glutamic acid. After subsequent neutralization, they have the general structure:

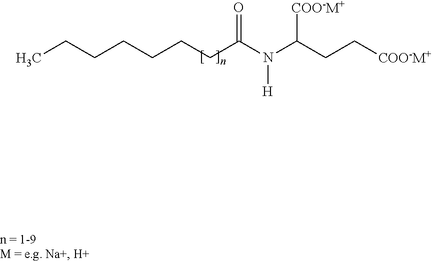

As representatives of this group, examples include, but are not limited to, the following raw materials with their INCI names: Sodium Cocoyl Glutamate, Sodium Lauroyl Glutamate, Disodium Cocoyl Glutamate.

In accordance with the present invention, anionic surfactants are combined with amphoteric surfactants. The proportion of the latter surfactants is typically about 0.1 to 20, preferably 1 to 15 and especially 2 to 10% by weight, wherein the percentages by weight refer to the total formulation.

The amphoteric surfactants according to the invention from the group of acylamidoalkyl betaines have the following general structure:

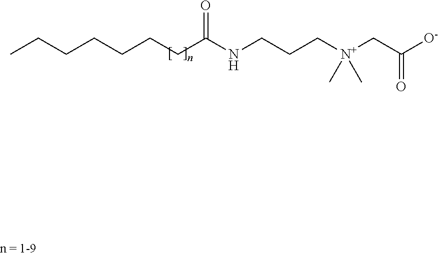

As representatives of this group, examples include, but are not limited to, the following raw materials with their INCI names: Cocoamidopropylbetaine, Lauramidopropylbetaine.

The amphoteric surfactants according to the invention from the group of alkyl betaines have the following general structure:

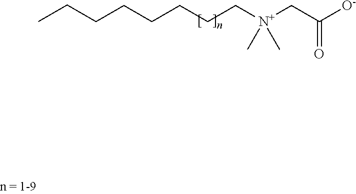

As representatives of this group, examples include, but are not limited to, the following raw materials with their INCI names: Coco Betaine, Cetyl Betaine.

The aforementioned combinations of washing-active substances may be stabilized against microbiological contamination in accordance with the invention with the aid of 2-methyl-1,3-propanediol. The 2-methyl-1,3-propanediol can be directly incorporated into the surfactant mixtures in this case and its application concentration should be between 0.1-10, preferably 1-8 and particularly preferably 2-7% by weight, wherein the percentages by weight refer to the total formulation.

Formulations according to the invention are further characterized in that they have a pH of 6 to 10, preferably 6 to 9.5, more preferably 6 to 9.

The “pH” in connection with the present invention is defined as the value which is measured at 25° C. using a calibrated pH electrode in accordance with ISO 4319 (1977).

Formulations according to the invention comprise no further preservatives selected from the following group:
authorized preservatives cited in Annex V of Regulation (EC) No. 1223/2009, and
multifunctional additives selected from the group of linear or branched, saturated or unsaturated aliphatic or aromatic hydroxamic acids having a number of 6-16 carbon atoms and aromatic alcohols of the general structure

Ar—(CH2)n-OH

where n=2-4.

REGULATION (EC) No. 1223/2009 OF THE EUROPEAN PARLIAMENT AND OF THE COUNCIL of 30 Nov. 2009 on cosmetic compositions includes Annex V published in the official journal of the European Union, L 342/59 of 22 Dec. 2009.

Annex V of this regulation is expressly incorporated by way of reference in the subject matter and disclosure content of the present patent application.

Annex V of Regulation (EC) No. 1223/2009 in the context of the present invention cites as authorized preservatives benzoic acid and sodium salt thereof and other salts of benzoic acid and benzoic esters, propionic acid and salts thereof, salicylic acid and salts thereof, 2,4-hexadienoic acid and salts thereof, formaldehyde and paraformaldehyde, 2-hydroxybiphenyl and salts thereof, zinc pyrithione, inorganic sulphites and bisulphites, chlorobutanol, 4-hydroxybenzoic acid, salts and esters thereof, 3-acetyl-6-methyl-2,4(3H)-pyrandione and salts thereof, formic acid and sodium salt thereof, 1,6-bis(4-amidino-2-bromophenoxy)-n-hexane (dibromohexamidine) and salts thereof including isethionate, thiomersal, phenylmercury and salts thereof including borate, 10-undecylenic acid and salts thereof, 5-pyrimidinamine, 1,3-bis(2-ethylhexyl)hexahydro-5-methyl, 5-bromo-5-nitro-1,3-dioxane, bronopol, 2,4-dichlorobenzyl alcohol, 1-(4-chlorophenyl)-3-(3,4-dichlorophenyl)urea, chlorocresol, 5-chloro-2-(2,4-dichlorophenoxy)phenol, chloroxylenol, N,N″-methylenebis[N′-[3-(hydroxymethyl)-2,5-dioxoimidazolidin-4-yl]urea, poly(methylene), α,ω-bis[[[(aminoiminomethyl)amino]iminomethyl]amino]-, dihydrochloride, 2-phenoxyethanol, methenamine, 1-(3-chloroallyl)-3,5,7-triaza-1-azoniaadamantane chloride, 1-(4-chlorophenoxy)-1-(1H-imidazol-1-yl)-3,3-dimethyl-2-butanone, 1,3-bis(hydroxymethyl)-5,5-dimethyl-2,4-imidazolidinedione, benzyl alcohol, 1-hydroxy-4-methyl-6-(2,4,4-trimethylpentyl)-2-pyridone and monoethanolamine salt thereof, 2,2′-methylenebis(6-bromo-4-chlorophenol) (bromochlorophen), 3-methyl-4-(1-methylethyl)phenol, mixture of 5-chloro-2-methyl-3(2H)-isothiazolone and 2-methyl-3(2H)-isothiazolone with magnesium chloride and magnesium nitrate, chlorophene, 2-chloroacetamide, N,N″-bis(4-chlorophenyl)-3,12-diimino-2,4,11,13-tetraazatetradecanediimidamide, acetate, gluconate and hydrochloride thereof, 3-phenoxy-1-propanol, alkyl(C12-22)trimethylammonium bromide and chloride, 4,4-dimethyl-1,3-oxazolidine, N-hydroxymethyl-N-[1,3-di(hydroxymethyl)-2,5-dioxoimidazolidinyl-4-yl]-TN-hydroxymethylurea, benzolcarboximidamide, 4,4′-(1,6-hexanediylbis(oxy))bis and salts thereof including isethionate and [symbol] -hydroxybenzoate, glutaraldehyde (1,5-pentanedial), 5-ethyl-3,7-dioxa-1-azabicyclo [3.3.0] octane, 3-(p-chlorophenoxy)-1,2-propanediol, sodium hydroxymethylamino acetate, silver chloride applied to titanium dioxide, benzenemethanaminium, N,N-dimethyl-N-[2-[2-[4-(1,1,3,3,-tetramethylbutyl)phenoxy]ethoxy]ethyl], chloride, benzalkonium chloride, bromide and saccharinate, phenylmethoxymethanol, 3-iodo-2-propynyl butylcarbamate, 2-methyl-2H-isothiazol-3-one,

wherein the term “salts” are understood to mean the salts of the cations sodium, potassium, calcium, magnesium, ammonium and ethanolamine and salts of the anions chloride, bromide, sulphate, acetate and the term “esters” are understood to mean methyl, ethyl, propyl, isopropyl, butyl, isobutyl and phenyl esters.

In the prior art, in addition to traditional preservatives according to Annex V of the Regulation (EC) No. 1223/2009, multifunctional additives are increasingly used which, in addition to their antimicrobial effect, have additional cosmetic, e.g. complex-forming or fragrance properties. Such multifunctional substances may also contribute to the biological stabilization of a cosmetic product. Formulations according to the invention dispense with the presence of linear or branched, saturated or unsaturated aliphatic hydroxamic acids having 6-16 carbon atoms such as caprylohydroxamic acid, and also aromatic alcohols of the general structure Ar—(CH2)n-OH where n=2-4.

A formulation according to the invention preferably comprises no further preservatives or microbiocides.

In addition to the components mentioned in the example formulations, formulations according to the invention comprise further raw materials such as those typically used in surfactant-containing formulations. If these formulations are cosmetic products for cleansing skin and/or hair, these may be, for example, moisture-regulating and wetting agents, additional surfactants, pearlescent waxes, consistency regulators, thickeners, conditioners, silicone compounds, antidandruff agents, complexing agents, film formers, swelling agents, hydrotropes, perfume oils, dyes etc., which are listed in the examples below.

Moisture-Regulating and Wetting Agents

Useful moisture-regulating and wetting agents are principally linear or branched polyols having a chain length of 4 to 12 carbon atoms, e.g. pentylene glycol, 1,2-hexanediol, heptylene glycol, caprylyl glycol, hexylene glycol, or any mixtures of linear or branched polyols. Monoglycerides of fatty acids having a chain length of 4-12 carbon atoms, e.g. glyceryl caprate or glycerol ethers of linear or branched alcohols having 4-8 carbon atoms, e.g. ethylhexylglycerin, are used for this purpose and may be present alone or in combination in formulations according to the invention.

Surfactants

In addition to the surfactants listed according to the invention, further anionic, non-ionic, cationic and/or amphoteric or zwitterionic surfactants may be present as surface-active substances.

Typical examples of further anionic surfactants are soaps, alkylbenzene sulphonates, alkane sulphonates, olefin sulphonates, alkyl ether sulphonates, glycerol ether sulphonates, α-methyl ester sulphonates, sulpho fatty acids, glycerol ether sulphates, fatty acid ether sulphates, hydroxyl-mixed ether sulphates, monoglyceride (ether) sulphates, fatty acid amide (ether) sulphates, mono- and dialkylsulphosuccinates, mono- and dialkylsulphosuccinamates, sulphotriglycerides, amide soaps, ether carboxylic acids and salts thereof, fatty acid thionates, fatty acid sarcosinates, fatty acid taurides, N-acylamino acids, such as, for example, acyllactylates, acyltartrates and acylaspartates, alkyloligoglucoside sulphates, protein fatty acid condensates (especially wheat-based plant products) and alkyl (ether) phosphates. Where the anionic surfactants contain polyglycol ether chains, they may have a conventional distribution, but preferably a narrowed homologue distribution. Typical examples of non-ionic surfactants are fatty acid polyglyceryl esters, fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucuronic acid derivatives, fatty acid N-alkylglucamides, protein hydrolysates (particularly wheat-based plant products), polyol fatty acid esters, sugar esters, sorbitan esters, polysorbates and amine oxides. Where the nonionic surfactants contain polyglycol ether chains, they may have a conventional homologue distribution, but preferably a narrowed homologue distribution. Typical examples of particularly suitable mild, i.e. particularly skin-compatible, surfactants are fatty alcohol polyglycol ether sulphates, monoglyceride sulphates, mono- and/or dialkyl sulphosuccinates, fatty acid isothionates, fatty acid sarcosinates, fatty acid taurides, fatty acid glutamates, α-olefinsulfonates, ether carboxylic acids, alkyl oligoglucosides, fatty acid glucamides, amphoacetals and/or protein fatty acid condensates, the latter preferably based on wheat proteins.

Pearlescent Waxes

Useful pearlescent waxes include, for example: alkylene glycol esters especially ethylene glycol distearate; fatty acid alkanolamides especially coconut fatty acid diethanolamide; partial glycerides especially stearic acid monoglyceride; esters of polybasic optionally hydroxyl-substituted carboxylic acids with fatty alcohols having 6 to 22 carbon atoms especially long-chain esters of tartaric acid; lipids such as fatty alcohols, fatty ketones, fatty aldehydes, fatty ethers and fatty carbonates having in total at least 24 carbon atoms especially laurone and distearyl ether; fatty acids such as stearic acid, hydroxystearic acid or behenic acid, ring-opening products of olefin epoxides having 12 to 22 carbon atoms with fatty alcohols having 12 to 22 carbon atoms and/or polyols having 2-15 carbon atoms and 2 to 10 hydroxyl groups and mixtures thereof.

Consistency Regulators and Thickeners

Additions of 2-methyl-1,3-propanediol may lead to a lowering of viscosity of the formulation. To restore the viscosity, in addition to increasing the electrolyte concentration by adding inorganic salts, consistency regulators and thickeners may also be used.

Useful consistency regulators are alkyl oligoglucosides, alone or in combination with fatty acid monoglycerides. Further suitable thickeners may be selected from the group of the Aerosil products (hydrophilic silicas), polysaccharides, particularly xanthan gum, guar-guar, agar-agar, alginates and tyloses, carboxymethyl cellulose and hydroxyethyl- and hydroxypropylcellulose, further higher molecular weight polyethylene glycol mono- and diesters of fatty acids, polyacrylates (e.g. Carbopole® and Permulene products from Goodrich; Synthalene® from Sigma; Keltrol products from Kelco; Sepigel products from Seppic; Salcare products from Allied Colloids), polyacrylamides, polymers, polyvinyl alcohol and polyvinylpyrrolidone. Further suitable surfactants are, for example, ethoxylated fatty acid glycerides or ethoxylated alkylglucose fatty acid esters, of which in particular PEG-120 methylglucose dioleate (Antil 127) has proved its worth for increasing viscosity.

Conditioners

Cationic polymers are frequently used as conditioners in products for cleansing and care of skin or hair. Suitable cationic polymers are, for example, cationic cellulose derivatives such as polyquaternium-10, cationic starches or mixtures thereof such as are supplied, for example, by Dr. Straetmans GmbH under the names Amylomer and Symbio®quat, copolymers of diallylammonium salts and acrylamides, quaternized vinylpyrrolidone/vinylimidazole polymers, condensation products of polyglycols and amines, quaternized collagen polypeptides such as lauryldimonium hydroxypropyl hydrolyzed collagen, quaternized wheat polypeptides, polyethyleneimine, cationic silicone polymers such as amodimethicone, copolymers of adipic acid and dimethylaminohydroxypropyldiethylenetriamine, copolymers of acrylic acid with dimethyldiallylammonium chloride, polyaminopolyamides and crosslinked water-soluble polymers thereof, cationic chitin derivatives such as quaternized chitosan, optionally distributed in microcrystalline form, condensation products of dihaloalkylene such as dibromobutane with bisdialkylamines such as bisdimethylamino-1,3-propane, cationic guar gum, and also quaternized ammonium salt polymers.

Suitable anionic, zwitterionic, amphoteric and non-ionic polymers are, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, non-crosslinked polyacrylic acids and polyacrylic acids crosslinked with polyols, acrylamidopropyltrimethylammonium chloride/acrylate copolymers, octylacrylamide/methyl methacrylate/tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinylcaprolactam terpolymers and also optionally derivatized cellulose ethers and silicones.

Silicone Compounds

Silicone compounds together with quaternized polymers form so-called coacervates which contribute to improvement in combability and shine in products for cleansing hair. Suitable silicone compounds are, for example, dimethylpolysiloxanes, methylphenylpolysiloxanes, cyclic silicones and also amino-, fatty acid-, alcohol-, polyether-, epoxy-, fluoro-, glycoside- and/or alkyl-modified silicone compounds, which may be present at room temperature both as liquid and in the form of resin. Further suitable are simethicones which take the form of mixtures of dimethicones having an average chain length of 200 to 300 dimethylsiloxane units and hydrogenated silicates.

Film Formers

Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid and salts thereof and similar compounds.

Antidandruff Active Ingredients

Useful antidandruff active ingredients, in addition to lactylates of fatty acids with a chain length of 8-14 carbon atoms, are piroctone olamine, climbazole, ketoconazole, elubiol, selenium disulphide, colloidal sulphur, sulphur polyethylene glycol sorbitan monooleate, sulphur ricinol polyethoxylate, sulphur tar distillates, undexylenic acid monoethanolamide sulphosuccinate Na salt, zinc pyrithione, aluminum pyrithione and magnesium pyrithione/dipyrithione magnesium sulphate.

Perfume Oils and Aromas

Perfume oils include mixtures of natural and synthetic odorants. Natural fragrances are extracts from flowers (lily, lavender, rose, jasmine, neroli, ylang ylang), stems and leaves (geranium, patchouli, petitgrain), fruits (anise, coriander, caraway, juniper), fruit peels (bergamot, lemon, orange), roots (mace, angelica, celery, cardamom, costus, iris, calmus), woods (pinewood, sandalwood, guaiac, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Additionally useful are animal raw materials, for example civet and castoreum. Typical synthetic odorant compounds are products of the ester, ether, aldehyde, ketone, alcohol and hydrocarbon type. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexylpropionate, styrallyl propionate and benzyl salicylate. The ethers include, for example, benzyl ethyl ether; the aldehydes include, for example, the linear alkanals having 8 to 18 carbon atoms, citral, citronellal, citronellyloxyacetaldehyde, cyclamen aldehyde, hydroxycitronellal, lilial and bourgeonal; the ketones include, for example, the ionones, α-isomethylionone and methyl cedryl ketone; the alcohols include anethole, citronellol, eugenol, isoeugenol, geraniol, linalool, phenylethyl alcohol and terpineol; the hydrocarbons include principally the terpenes and balsams. Preference is given, however, to using mixtures of different odorants which together produce a pleasing fragrance note. Suitable perfume oils are also essential oils of relatively low volatility which are usually used as aroma components, for example sage oil, camomile oil, clove oil, melissa oil, mint oil, cinnamon leaf oil, lime blossom oil, juniper berry oil, vetiver oil, olibanum oil, galbanum oil, labdanum oil and lavender oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, Boisambrene Forte, ambroxan, indole, Hedione, Sandelice, lemon oil, mandarin oil, orange oil, allyl amyl glycolate, cyclovertal, lavender oil, clary sage oil, □-damascone, geranium oil bourbon, cyclohexyl salicylate, Vertofix Coeur, Iso-E-Super, Fixolide NP, Evernyl, iraldein gamma, phenylacetic acid, geranyl acetate, benzyl acetate, rose oxide, Romilat, Irotyl and Floramat, alone or in mixtures.

From the group of organic acids, mention may be made of levulinic acid, cinnamic acid or anisic acid for example.

Useful aromas are, for example, peppermint oil, spearmint oil, anise oil, star anise oil, caraway oil, eucalyptus oil, fennel oil, lemon oil, wintergreen oil, oil of cloves, menthol and the like.

Some aromas and fragrances may themselves have antimicrobial properties or may enhance synergistically the antimicrobial effect in combination with preservatives or preservative auxiliaries. The synergistic effects of these fragrances are explicitly incorporated in this patent specification.

Dyes

Dyes which may be used are the substances approved and suitable for cosmetic purposes. Examples are cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). As a luminescent dye, it is also possible for luminol to be present. These dyes are typically used at concentrations of 0.001 to 0.1% by weight, based on the overall formulation.

The invention further provides for the use of 2-methyl-1,3-propanediol as sole biocide for microbiological stabilization of an aqueous formulation comprising:

a) at least one anionic surfactant selected from the group of alkyl sulphates, alkyl ether sulphates and/or acyl glutamates,

b) at least one amphoteric surfactant selected from the group of alkyl betaines and/or amidoalkyl betaines.

The formulation “sole biocide”, in addition to 2-methyl-1,3-propanediol, provides that further multifunctional raw materials may be present in the formulation which, in addition to another function, likewise may contribute to the inhibition of microbial growth. If these are not explicitly excluded in any of the claims, their subject matter in the formulations according to the invention is claimed.

The formulations according to the invention may be prepared according to the prior art and, as demonstrated in the following test formulations, have an excellent stability with respect to the relevant surfactant-containing, particularly cosmetic, formulations and microbes used in a microbiological contamination test.

Relevant microbes are particularly Staphylococcus aureus, Pseudomonas aeruginosa, Escherichia coli, Candida albicans and/or Aspergillus brasiliensis.

The efficacy clearly exceeds the effect of a preservation system of the prior art (surfactant base 2b), as the direct comparison shows. The following examples are intended to illustrate the present invention without limiting it.

### Comparative Example 1

An O/W emulsion was prepared corresponding to the technical teaching of FR 2780283 A1, but without comprising the complexing agent Na4 EDTA (all figures are parts by weight):

O/W Emulsion 1, pH = 6.5

| Phase | Raw material | INCI (EU) | % |
|---|---|---|---|
| A | Deionised Water | Aqua | 66.80 |
| | dermofeel ® PA-3 | Sodium Phytate; Aqua; | 0.10 |
| | | Alcohol | |
| | MP-Diol Glycol | Methylpropanediol | 5.00 |
| | Glycerol 99.5% | Glycerol | 5.00 |
| A1 | Keltrol CG-RD | Xanthan Gum | 0.30 |
| B | dermofeel ® GSC | Glyceryl Stearate Citrate | 3.50 |
| | Miglyol 812 N | Caprylic/Capric Triglyceride | 6.00 |
| | Phytosqualane, veg. | Squalane | 6.00 |
| | grade | | |
| | Sunflower Oil | Helianthus Annuus Seed Oil | 5.00 |
| | Lanette O | Cetearyl Alcohol | 2.00 |
| | Perf. Nat. Sunny | Perfume | 0.30 |
| | Pomegranate P0250284 | | |
| | | | 100.0 |

A preservative contamination test according to Pharm. Eur. 2014, 5.1.3 showed:

| O/W | | | | | |
|---|---|---|---|---|---|
| Emulsion 1, | | | | | |
| pH = 6.5 | 0 days | 2 days | 7 days | 14 days | 28 days |
| Staphyl- | 3.9 × 10⁵ | 1.6 × 10⁵ | 2.1 × 10⁵ | 2.0 × 10⁵ | 1.3 × 10⁵ |
| ococcus | | | | | |
| aureus | | | | | |
| Pseudo- | 5.8 × 10⁵ | 1.1 × 10⁵ | 8.0 × 10⁴ | 6.2 × 10⁴ | 1.2 × 10⁴ |
| monas | | | | | |
| aeruginosa | | | | | |
| Escherichia | 6.1 × 10⁵ | 8.0 × 10⁴ | 1.2 × 10⁵ | 1.1 × 10⁵ | 2.3 × 10⁴ |
| coli | | | | | |
| Candida | 3.4 × 10⁵ | 2.2 × 10⁵ | 2.9 × 10⁵ | 3.6 × 10⁵ | 5.5 × 10⁵ |
| albicans | | | | | |
| Aspergillus | 3.2 × 10⁵ | 1.0 × 10⁵ | 8.4 × 10⁴ | 6.1 × 10⁴ | 8.0 × 10⁴ |
| brasiliensis | | | | | |

It is recognized that use of 2-methyl-1,3-propanediol does not automatically and inevitably show the desired microbiocidal effect, rather the inventive combination claimed is essential for the success of the invention.

### Comparative Example 2 and Examples 1 to 3

The formulations shown in the following table were prepared. Surfactant base 2b is comparative example 2, surfactant base 1, 2a and 3 are inventive examples 1 to 3. The percentages are parts by weight.

| Raw | | Surfactant | Surfactant | Surfactant | Surfactant |
|---|---|---|---|---|---|
| material | INCI | base 1 | base 2a | base 2b | base 3 |
| Texapon N | Sodium Laureth | 12.0% | 10.0% | 10.0% | — |
| 70 | Sulfate; Aqua | | | | |
| Tego Betain | Cocoamidopropyl | 0.08 | 0.07 | 0.07 | 9.0% |
| F 50 | Betaine; Aqua | | | | |
| Amisoft | Disodium Cocoyl | — | 5.0% | 5.0% | 5.0% |
| CS-22 | Glutamate; | | | | |
| | Sodium Cocoyl | | | | |
| | Glutamate; Aqua | | | | |
| Plantacare | Lauryl Glucoside; | — | 5.0% | 5.0% | 13.5% |
| 1200 UP | Aqua | | | | |
| Lamesoft | Coco Glucoside, | — | — | — | 2.0% |
| PO 65 | Glyceryl Oleate | | | | |
| MP-Diol | Methylpropanediol | 5.0% | 3.0% | | 5.0% |
| Glycol | | | | | |
| Verstatil PC | Phenoxyethanol, | — | — | 1.0% | — |
| | Caprylyl Glycol | | | | |
| Water | Aqua | to 100% | to 100% | | to 100% |

| | | | | |
|---|---|---|---|---|
| pH | 6.5 | 7.5 | 7.0 | 6.5 |

All tests were carried out according to the requirements of a preservative contamination test according to Pharm. Eur. 2014, 5.1.3.

| | 0 days | 2 days | 7 days | 14 days | 28 days |
|---|---|---|---|---|---|
| Surfactant base 1 | | | | | |
| Staphylococcus aureus | 3.9 × 10⁵ | <10 | <10 | <10 | <10 |
| Pseudomonas aeruginosa | 5.1 × 10⁵ | <10 | <10 | <10 | <10 |
| Escherichia coli | 3.7 × 10⁵ | <10 | <10 | <10 | <10 |
| Candida albicans | 7.0 × 10⁵ | <10 | <10 | <10 | <10 |
| Aspergillus brasiliensis | 3.6 × 10⁵ | 7.0 × 10⁴ | 1.7 × 10⁴ | <10 | <10 |
| Surfactant base 2a | | | | | |
| Staphylococcus aureus | 3.9 × 10⁵ | <10 | <10 | <10 | <10 |
| Pseudomonas aeruginosa | 5.1 × 10⁵ | <10 | <10 | <10 | <10 |
| Escherichia coli | 3.7 × 10⁵ | <10 | <10 | <10 | <10 |
| Candida albicans | 7.0 × 10⁵ | <10 | <10 | <10 | <10 |
| Aspergillus brasiliensis | 3.6 × 10⁵ | 4.8 × 10⁴ | 10 | <10 | <10 |
| Surfactant base 2b | | | | | |
| Staphylococcus aureus | 6.3 × 10⁵ | 8.0 × 10⁴ | 3.7 × 10⁴ | 2.2 × 10⁴ | 4.0 × 10³ |
| Pseudomonas aeruginosa | 7.0 × 10⁵ | 1.2 × 10⁵ | 1.2 × 10⁵ | 1.2 × 10⁵ | 1.0 × 10⁵ |
| Escherichia coli | 9.0 × 10⁵ | 8.0 × 10⁴ | 1.0 × 10⁵ | 1.0 × 10⁵ | 1.0 × 10⁵ |
| Candida albicans | 7.2 × 10⁵ | 4.0 × 10⁴ | 6.0 × 10⁴ | 6.0 × 10⁴ | 1.8 × 10⁴ |
| Aspergillus brasiliensis | 3.0 × 10⁵ | 6.0 × 10⁴ | 6.4 × 10⁴ | 6.4 × 10⁴ | 4.0 × 10⁴ |
| Surfactant base 3 | | | | | |
| Staphylococcus aureus | 3.9 × 10⁵ | <10 | <10 | <10 | <10 |
| Pseudomonas aeruginosa | 5.1 × 10⁵ | <10 | <10 | <10 | <10 |
| Escherichia coli | 3.7 × 10⁵ | <10 | <10 | <10 | <10 |
| Candida albicans | 7.0 × 10⁵ | <10 | <10 | <10 | <10 |
| Aspergillus brasiliensis | 3.6 × 10⁵ | 4.6 × 10⁴ | <10 | <10 | <10 |

The tests show the superiority of the formulations according to the invention.

1. An aqueous formulation comprising
a) an anionic surfactant selected from the group of alkyl sulphates, alkyl ether sulphates and/or acyl glutamates,
b) an amphoteric surfactant selected from the group consisting of alkyl betaines and/or amidoalkyl betaines,
c) 2-methyl-1,3-propanediol,
wherein
d) said formulation has a pH of 6 to 10,
e) comprises no further preservatives for microbiological stabilization selected from the group consisting of:
e1) authorized preservatives cited in Annex V of Regulation (EC) No. 1223/2009, and
e2) multifunctional additives selected from the group of linear or branched, saturated or unsaturated aliphatic or aromatic hydroxamic acids having a number of 6-16 carbon atoms and aromatic alcohols of the general structure
Ar—(CH2)n-OH
where n=2-4.

2. The aqueous formulation according to claim 1, wherein the anionic surfactant according to a) is present at a concentration of 0.1 to 30% by weight.

3. The aqueous formulation according to claim 2, wherein the anionic surfactant according to a) is selected from the group consisting of sodium lauryl sulphate, ammonium lauryl sulphate, sodium laureth sulphate, sodium cocoyl glutamate and disodium cocoyl glutamate.

4. The aqueous formulation according to claim 1, wherein the amphoteric surfactant according to b) is present at a concentration of 0.1 to 20% by weight.

5. The aqueous formulation according to claim 4, wherein the amphoteric surfactant according to b) is a quaternary surfactant selected from the group consisting of cocoamidopropyl betaine and coco betaine.

6. The aqueous formulation according to claim 1, wherein 2-methyl-1,3-propanediol is present at a concentration of 0.1 to 10% by weight.

7. The aqueous formulation according to claim 1, wherein 2-methyl-1,3-propanediol is used for microbiological stabilization of the formulation against Staphylococcus aureus, Pseudomonas aeruginosa, Escherichia coli, Candida albicans and/or Aspergillus brasiliensis.

8. The aqueous formulation according to claim 1, wherein the pH is from 6 to 9.5.

9. A cosmetic product for cleansing and/or care of skin and/or hair, wherein said product comprises a formulation according claim 1.

10. A formulation according to claim 1 in a cosmetic product for cleansing and/or care of skin and/or hair.

11. An aqueous formulation comprising 2-methyl-1,3-propanediol as sole biocide for microbiological stabilization:
a) at least one anionic surfactant selected from the group of alkyl sulphates, alkyl ether sulphates and/or acyl glutamates,
b) at least one amphoteric surfactant selected from the group of alkyl betaines and/or amidoalkyl betaines.

12-13. (canceled)

14. The aqueous formulation according to claim 1, wherein the anionic surfactant according to a) is present at a concentration of 2 to 25% by weight.

15. The aqueous formulation according to claim 1, wherein the anionic surfactant according to a) is present at a concentration of 5 to 20% by weight.

16. The aqueous formulation according to claim 1, wherein the amphoteric surfactant according to b) is present at a concentration of 1 to 15% by weight.

17. The aqueous formulation according to claim 2, wherein the amphoteric surfactant according to b) is present at a concentration of 1 to 15% by weight.

18. The aqueous formulation according to claim 3, wherein the amphoteric surfactant according to b) is present at a concentration of 1 to 15% by weight.

19. The aqueous formulation according to claim 1, wherein the 2-methyl-1,3-propanediol is present at a concentration of 1 to 8% by weight.

20. The aqueous formulation according to claim 1, wherein the pH is from 6 to 9.
